(19) United States
(12) Patent Application Publication
(10) Pub. No.: US 2024/0400530 A1
(43) Pub. Date: Dec. 5, 2024

(54) METHODS FOR PREPARING BUTENOLIDES

(71) Applicant: The Hong Kong University of Science and Technology, Hong Kong (CN)

(72) Inventors: Rongbiao TONG, Hong Kong (CN); Yuen Tsz CHEUNG, Hong Kong (CN)

(21) Appl. No.: 18/635,130

(22) Filed: Apr. 15, 2024

Related U.S. Application Data
(60) Provisional application No. 63/504,792, filed on May 30, 2023.

(51) Int. Cl. C07D 307/33

(52) U.S. Cl. CPC C07D 307/33

(57) ABSTRACT

A method useful for preparing butenolides in high yield, the method comprising: contacting an aldehyde, malonic acid, and an organic base thereby forming a Knoevenagel condensation product; and contacting the Knoevenagel condensation product, an oxidant, a metal halide, and optionally an inorganic base thereby forming the butenolide.

### CROSS-REFERENCE TO RELATED APPLICATIONS

The present application claims priority from U.S. Provisional Patent Application No. 63/504,792, filed on May 30, 2023, which is hereby incorporated by reference in its entirety.

### BACKGROUND

Biofouling poses significant challenges to the marine industry. Microorganisms and invertebrates responsible for biofouling reduce the longevity of submerged surfaces and create hydrodynamic faults, including increased drag and reduced fuel efficiency, to vessel hulls. At the same time, the spread of non-indigenous species through maritime activities may imperil ecosystem integrity. Defouling commonly necessitates dry dock abrasive blasting, which can prove arduous and lengthy. Application of antifouling paint is a common means of biofouling prevention.

Copper-based paints have largely replaced tributyltin (TBT) as a biocide in antifouling paint since their discovery in the 1960s due to the acute toxicity of TBT. Later, the negative impacts of copper and other early organic biocides on the environment were discovered, which increased the need to develop new non-toxic antifouling paints. Marine natural products, potential candidates of non-toxic antifouling agents, have been widely investigated, because of the rare observation of biofouling on the surface of marine organisms. In 2010, a series of α,β-unsaturated lactones isolated from marine bacterium Streptomyces albidoflavus strain UST040711-291 were shown to exhibit strong antifouling activity. After further investigation and structural optimization, natural product derivative butenolide, C4 substituted lactone with a long linear aliphatic chain, was found to be the most active analaog with the least toxicity. The excellent field results clearly demonstrate the potential of butenolide as a new type of antifouling agent. However, butenolides are only isolated in microscopic amounts from marine organisms, which does not yield sufficient quantities for industrial applications. Consequently, there is an urgent need for efficient large-scale chemical synthesis of butenolides for possible commercialization.

In the past half century, efforts were made to develop the chemical synthesis of butenolides. In 1977, Tishler reported a reductive lactonization of propiolic acid derivatives with Lindlar catalyst, which afforded a cis-olefin intermediate, which then underwent lactonization to give the desired butenolides. Another common synthetic approach towards butenolides involved a two-steps sequence: 1) oxidative lactonization and 2) elimination. The β,γ-unsaturated acids can be prepared by Knoevenagel condensation of an aldehyde and malonic acid and then oxidized using different types of oxidants, such as hypervalent selenium compounds, hypervalent iodine compounds and reactive halogen species (RHS), to afford the desired β-substituted-γ-lactones. Use of selenide and sulfide lactones requires an additional oxidation step to convert the β-substituent to a better leaving group. However, other intermediates can be directly subjected to β-elimination in by reaction with the appropriate base. For example, hypervalent iodine oxidants or RHS can furnish sulfonate, carboxylate or halide substituted lactone with good leaving groups for elimination. Adding base after the completion of oxidative lactonization enables a one-pot synthesis of butenolides. Koser reported an example of a one-pot synthetic method utilizing [hydroxy(tosyloxy)iodo]benzene as the oxidant for the synthesis of β-substituted tosyloxylactones. Further elimination of tosylic acid by DBU affords the desired butenolide. However, low yield and difficult isolation are the main drawbacks that prevent large-scale synthesis using the aforementioned reaction sequences of butenolides.

Later, two patents targeting the industrial synthesis of butenolides were reported with a similar approach, the two-step sequence involves bromolactionization and elimination. The main difference was the source of reactive bromine species (RBS). The former patent method by Tian (2018) utilized the in situ generation of RBS from DMSO and oxalyl bromide [(COBr)₂], while the latter one from Shi (2021) directly involved the use of N-bromosuccinimide (NBS) as the RBS. Distinct from the former one requiring addition of base K₂CO₃ for the elimination of HBr, succinimide anion generated from NBS was basic to promote the elimination without additional base. A simpler procedure with lower cost improved the competitiveness of this method. However, the use of toxic and moisture-sensitive reactive oxalyl bromide and NBS are the practical disadvantages in industrial synthesis. Additionally, the above methods employ stoichiometic amount of organic oxidants and large amount of organic solvent and consequently generate substantial quantities of organic waste. Notably, extra purification was required to obtain the pure product. The total cost of synthetic butenolides is so high and losing competiveness. A green and cost effective synthetic route for butenolides is yet to be developed, which is the main work of this invention.

There is thus a need for improved methods for preparing butenolides that address at least some of the disadvantages noted above.

### SUMMARY

Provided herein is a method for preparing a butenolide derivatives in a two-step process: 1) Knoevenagel condensation of an aldehyde and malonic acid to produce a β,γ-unsaturated carboxylic acid; 2) oxidative cyclization of the β,γ-unsaturated carboxylic acid using a green oxidant (peroxide or oxone) and an alkaline halide. This new approach avoids the use of toxic stoichiometric oxidants, such as oxalyl bromide and NBS, which reduces waste and improves the ecofriendliness of the method. The ecofriendly nature of this approach using cheap green reagents without producing toxic organic wastes makes it advantageous and cost-effective for large scale production of butenolides as additives of antifouling paints.

Two green approaches for the generation of RHS, oxone-halide and hydrogen peroxide-halide, are used in the methods described herein. These two green approaches feature the use of inorganic oxidants with low environmental toxicity and high atom economy and only generate non-toxic byproducts (potassium sulfate and water), which holds great potential for large-scale synthesis. Described herein are ecofriendly methods for efficient synthesis of butenolides. Oxone and hydrogen peroxide are used to oxidize halide to generate reactive halogen species, which promote the halolactonization of β,γ-unsaturated acid intermediate. Subsequent elimination of HX can be performed with or without base to yield the butenolides in high yield (87%) and purity (purity >97% based on NMR). In a specific example, 415 grams of butenolide (2.66 mol) was prepared using hydrogen peroxide and a catalytic amount of halide. Additionally, the method described herein can advantageously improve the reaction conditions of Knoevenagel condensation by 1) increasing the reaction concentration almost ten fold and 2) recycling the organic solvent with distillation in vacuum. These improvements reduce both the cost and the waste.

In a first aspect, provided herein is a method of preparing a butenolide, the method comprising: contacting an aldehyde, malonic acid, and an organic base thereby forming a Knoevenagel condensation product; and contacting the Knoevenagel condensation product, an oxidant, a metal halide, and optionally an inorganic base thereby forming the butenolide.

In certain embodiments, the organic base is an organic amine, an organic amidine, or an organic guanidine.

In certain embodiments, the oxidant is a peroxide or oxone.

In certain embodiments, the metal halide is a Group I or Group II metal salt of chloride, bromide, or iodide.

In certain embodiments, the butenolide has Formula 1:

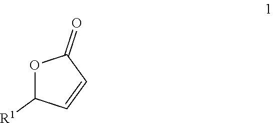

wherein R¹ is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —CN, —(C═O)OR, —OR, —O(C═O)R, —OSiR₃, —O(C═O)OR, —(C═O)NR₂, —(NR)(C═O)R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl.

In certain embodiments, the organic base is an organic amine.

In certain embodiments, the organic base is selected from the group consisting of diethyl amine, diisopropyl amine, pyrrolidine, piperidine, hexamethyleneimine, proline, morpholine, piperazine, imidazole, pyridine, triethylamine, N,N-diisopropylethylamine, N-ethyl pyrrolidine, N-nethyl morpholine, quinuclidine, and 1,4-diazabicyclo [2.2.2] octane.

In certain embodiments, the organic base comprises piperidine.

In certain embodiments, the step of contacting the aldehyde, malonic acid, and piperidine is conducted in a solvent comprising dimethyl sulfoxide, dimethyl formamide, or a mixture thereof; and piperidine is present at 0.5-3 mol % relative to the aldehyde.

In certain embodiments, the step of contacting the aldehyde, malonic acid, and piperidine is conducted in a solvent comprising dimethyl sulfoxide at 50-100° C.

In certain embodiments, the step of contacting the aldehyde, malonic acid, and piperidine is conducted in a flow chemistry reactor in a solvent comprising dimethyl sulfoxide, dimethyl formamide, or a mixture thereof at 100-140° C.

In certain embodiments, the flow chemistry reactor is operated at a flow rate of 0.4 mL/min to 6.0 mL/min.

In certain embodiments, the oxidant comprises oxone; the metal halide comprises a Group I metal salt of iodide; and the inorganic base comprises a Group I or Group II metal carbonate or bicarbonate.

In certain embodiments, the inorganic base comprises sodium carbonate, the metal halide comprises potassium iodide, and the step of contacting the Knoevenagel condensation product, oxone, the potassium iodide, and sodium carbonate is conducted in a solvent comprising acetonitrile and water in a volume ratio of 2:1 to 1:2, respectively.

In certain embodiments, the step of contacting the Knoevenagel condensation product, oxone, potassium iodide, and sodium carbonate is conducted at −10 to 23° C.

In certain embodiments, the oxidant comprises hydrogen peroxide; and the metal halide comprises a Group I or Group II metal bromide or iodide.

In certain embodiments, the metal halide comprises sodium iodide and the step of contacting the Knoevenagel condensation product, hydrogen peroxide, and sodium iodide is conducted in a solvent comprising acetonitrile and water in a volume ratio of 2:1 to 1:2, respectively.

In certain embodiments, the step of contacting the Knoevenagel condensation product, hydrogen peroxide, and sodium iodide is conducted at −10 to 23° C.

In certain embodiments, the method comprises: contacting an aldehyde having the formula R¹CH₂CHO, malonic acid and piperdine in a solvent comprising dimethyl sulfoxide at 50-100° C., wherein piperidine is present at 0.5-3 mol % relative to the aldehyde; or contacting an aldehyde having the formula R¹CH₂CHO, malonic acid, and piperdine in a solvent comprising dimethyl formamide in a flow chemistry reactor at a temperature of 100-140° C. and a flow rate of 0.4 mL/min to 6.0 mL/min; thereby forming the Knoevenagel condensation product; and contacting the Knoevenagel condensation product, oxone, potassium iodide, and sodium carbonate in a solvent comprising acetonitrile and water in a volume ratio of 2:1 to 1:2, respectively at −10 to 23° C.; or contacting the Knoevenagel condensation product, hydrogen peroxide, and sodium iodide in a solvent comprising acetonitrile and water in a volume ratio of 2:1 to 1:2, respectively at −10 to 23° C.; thereby forming the butenolide, wherein R¹ is is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —CN, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl.

In certain embodiments, the method comprises: contacting the aldehyde having the formula R¹CH₂CHO, malonic acid, and piperdine in a solvent comprising dimethyl formamide in a flow chemistry reactor at a temperature of 100-140° C. and a flow rate of 0.4 mL/min to 6.0 mL/min thereby forming the Knoevenagel condensation product; and contacting the Knoevenagel condensation product, oxone, potassium iodide, and sodium carbonate in a solvent comprising acetonitrile and water in a volume ratio of 2:1 to 1:2, respectively at −10 to 23° C.; or contacting the Knoevenagel condensation product, hydrogen peroxide, and sodium iodide in a solvent comprising acetonitrile and water in a volume ratio of 2:1 to 1:2, respectively at −10 to 23° C. thereby forming the butenolide.

### BRIEF DESCRIPTION OF THE DRAWINGS

The foregoing aspects and many of the attendant advantages of this invention will become more readily appreciated and understood by reference to the following detailed description, when taken in conjunction with the accompanying drawings.

FIG. 1 shows (a) a conventional method for preparing butenolides using Lindar catalyst; (b) oxidative lactonization; and (i and ii) additional oxidative lactonization methods involving hypervalent in iodine, oxalyl bromide, and NBS.

FIG. 2 shows a general reaction scheme for preparing butenolides in accordance with certain embodiments described herein involving Knoevenagel condensation and green oxidative lactonization and elimination.

FIG. 3 shows a table showing experimental results of Knoevenagel condensation reactions in which the equivalents and concentration of the malonic acid are varied.

FIG. 4 shows a table and graph showing experimental results of Knoevenagel condensation reactions conducted in a flow chemistry reactor in which the flow rate is varied.

FIG. 5 shows a table and graph showing experimental results of Knoevenagel condensation reactions conducted in a flow chemistry reactor in which reaction temperature is varied.

FIG. 6 shows a table and graph showing design of experiments (DOE) experimental results of Knoevenagel condensation reactions conducted in a flow chemistry reactor in which reaction temperature and flow rate are varied.

FIG. 7 shows optimal reaction conditions and yield of the Knoevenagel condensation prepared in a flow chemistry reactor in accordance with certain embodiments described herein.

FIG. 8 shows a table showing experimental results of the oxone/KBr mediated oxidative cyclization+in situ elimination reactions in which the acetonitrile to water solvent ratio is varied.

FIG. 9 shows a table showing experimental results of the oxone/KI mediated oxidative cyclization+in situ elimination reactions in which the KI equivalents and Na₂CO₃ equivalents are varied.

FIG. 10 shows a table showing experimental results of the H₂O₂/KI mediated oxidative cyclization+in situ elimination reactions in which the acetonitrile to water solvent ratio, NaI equivalents, and concentration of substrate are varied.

FIG. 11 shows optimal reaction conditions for the H₂O₂/KI mediated oxidative cyclization+in situ elimination starting from crude Knoevenagel condensation product used directly from the Knoevenagel condensation reactions without purification.

FIG. 12 shows the yield of butenolides (1) and (2) prepared in accordance with the methods described herein not involving flow chemistry as well as more complex hydroxy-substituted prepared over five steps.

FIG. 13 shows the yield of butenolide (1) prepared in accordance with the methods described herein involving flow chemistry on decagram scale.

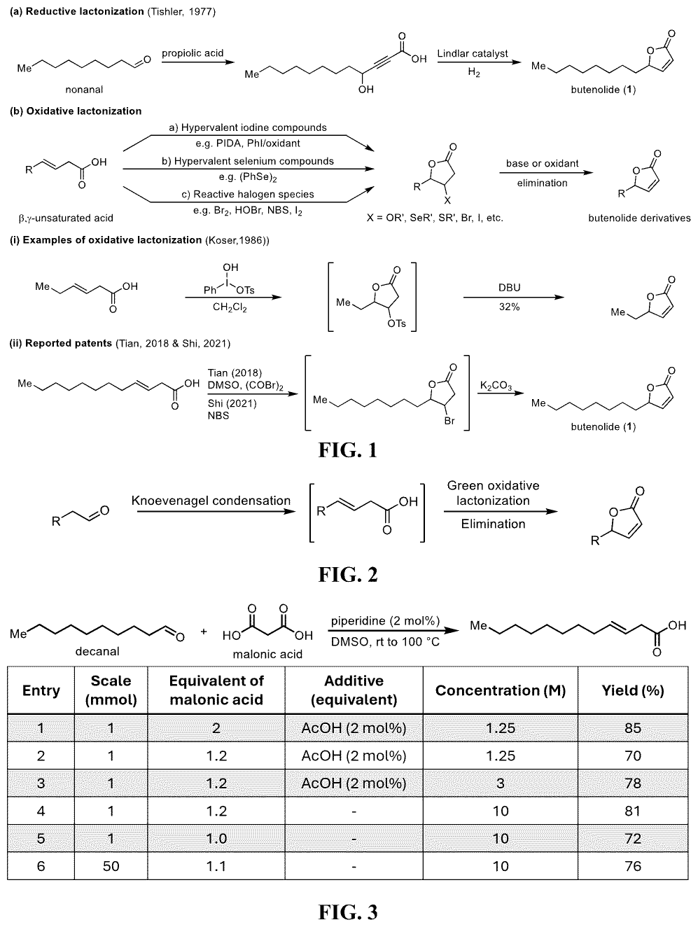

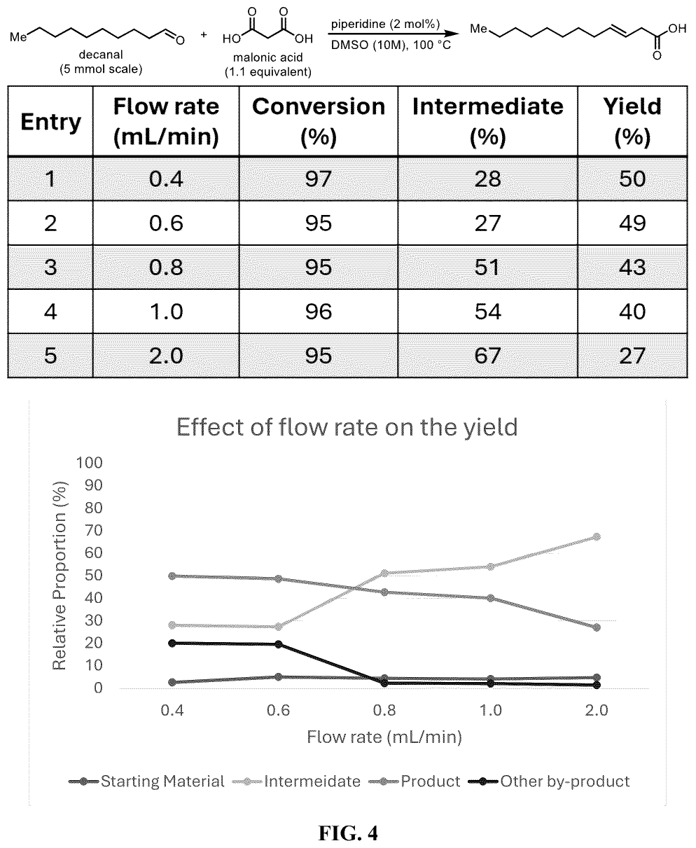

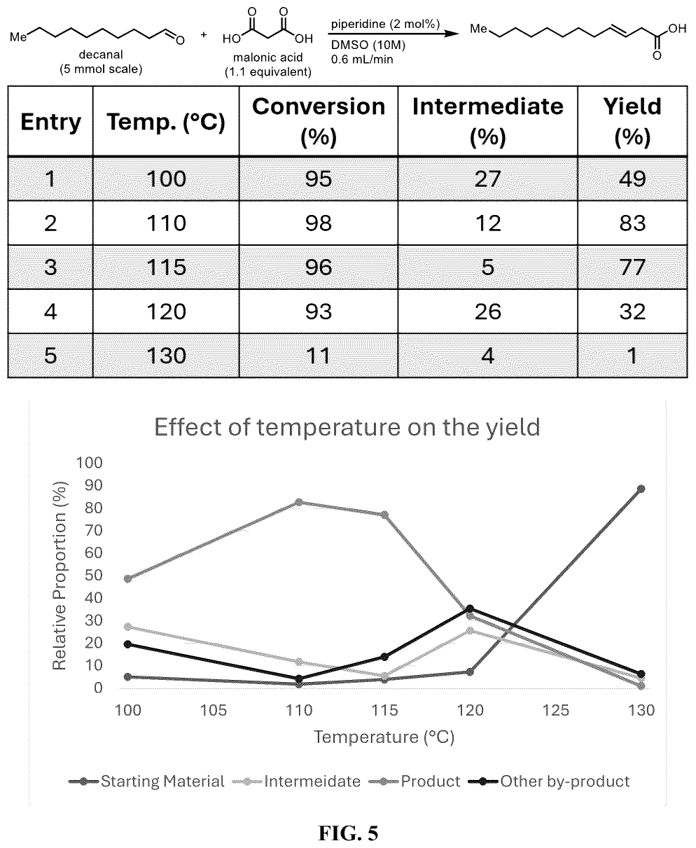

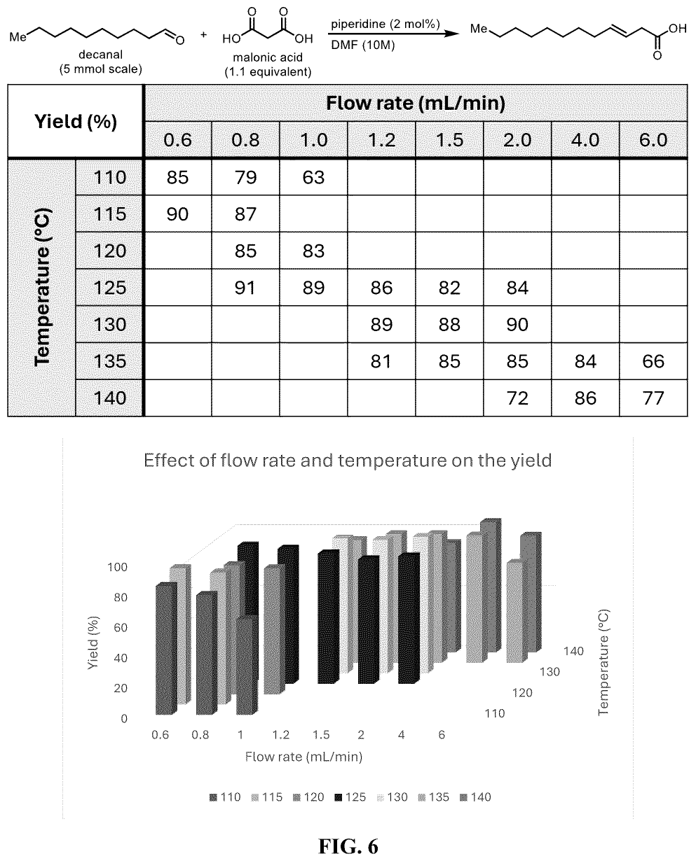

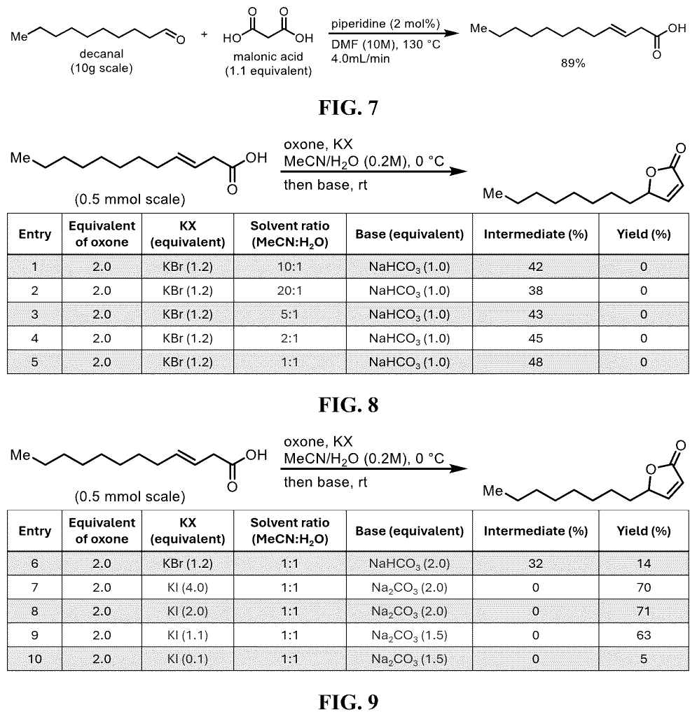

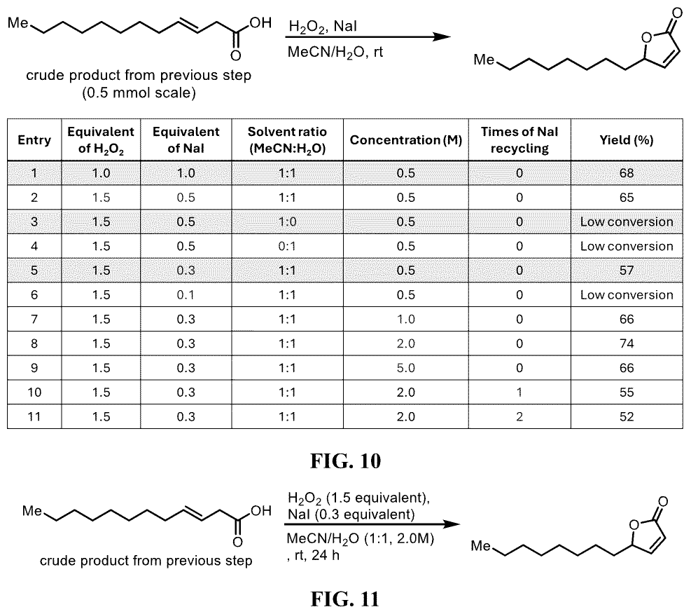

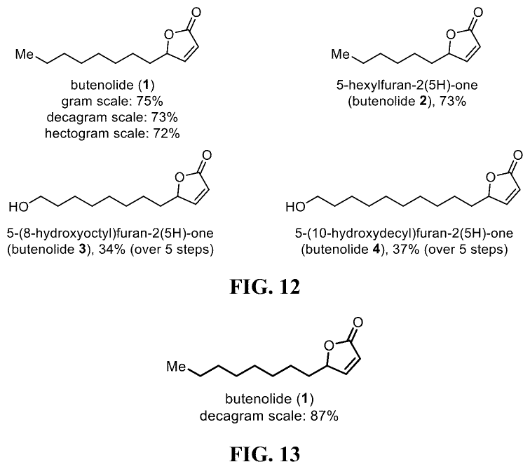

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

The term “composition” is intended to encompass a product comprising the specified ingredients in the specified amounts, as well as any product that results, directly or indirectly, from combinations of the specified ingredients in the specified amounts.

As used herein, “alkyl” refers to a straight-chain or branched saturated hydrocarbon group. Examples of alkyl groups include methyl-, ethyl-, propyl (e.g., n-propyl and isopropyl), butyl (e.g., n-butyl, iso-butyl, sec-butyl, tert-butyl), pentyl groups (e.g., 1-methylbutyl, 2-methylbutyl, iso-pentyl, tert-pentyl, 1,2-dimethylpropyl, neopentyl, and 1-ethylpropyl), hexyl groups, and the like. In various embodiments, an alkyl group can have 1 to 40 carbon atoms (i.e., C1-40 alkyl group), for example, 1-30 carbon atoms (i.e., C1-30 alkyl group). In certain embodiments, an alkyl group can have 1 to 6 carbon atoms, and can be referred to as a “lower alkyl group.” Examples of lower alkyl groups include methyl, ethyl, propyl (e.g., n-propyl and isopropyl), and butyl groups (e.g., n-butyl, isobutyl, sec-butyl, tert-butyl). In certain embodiments, alkyl groups can be optionally substituted as described herein. An alkyl group is generally not substituted with another alkyl group, an alkenyl group, or an alkynyl group.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C2-40 alkenyl group), for example, 2 to 20 carbon atoms (i.e., C2-20 alkenyl group). In certain embodiments, alkenyl groups can be substituted as described herein. An alkenyl group is generally not substituted with another alkenyl group, an alkyl group, or an alkynyl group.

As used herein, unless otherwise indicated, the term “alkynyl” includes alkyl groups as defined above having at least one carbon—carbon triple bond at some point in the alkyl chain.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

The term “aralkyl” is art-recognized and refers to an alkyl group substituted with an aryl group (e.g., an aromatic or heteroaromatic group).

The term “aryl” as used herein includes substituted or unsubstituted single-ring aromatic groups in which each atom of the ring is carbon. Preferably the ring is a 5-to 10-membered ring, more preferably a 6-to 10-membered ring or a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like. The aryl group can be optionally substituted. Exemplary substitution on an aryl group can include, for example, a halogen, a haloalkyl such as trifluoromethyl, a hydroxyl, a carbonyl (such as a carboxyl, an alkoxycarbonyl, a formyl, or an acyl such as an alkylC(O)), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a silyl ether, a sulfhydryl, an alkylthio, a sulfate, a sulfonate, a sulfamoyl, a sulfonamido, a sulfonyl, a heterocyclyl, an aralkyl, or an aromatic or heteroaromatic moiety. The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

As used herein, “heteroaryl” refers to an aromatic monocyclic ring system containing at least one ring heteroatom selected from oxygen (O), nitrogen (N), sulfur(S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide thiophene S-oxide, thiophene S,S-dioxide). Examples of heteroaryl groups include, for example, the 5-or 6-membered monocyclic and 5-6 bicyclic ring systems shown below: where T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In certain embodiments, heteroaryl groups can be substituted as described herein. In certain embodiments, heteroaryl groups can be optionally substituted.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulfur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

The term “optionally substituted” refers to a chemical group, such as alkyl, cycloalkyl, aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like.

The term “nitro” is art-recognized and refers to —NO₂; term “nitrile” is art-recognized and refers to —CN; the term “halogen” is art-recognized and refers to —F, —Cl, —Br or —I; and the term “hydroxyl” means —OH. “Halide” designates the corresponding anion of the halogens.

The present disclosure provides a method of preparing a butenolide, the method comprising: contacting an aldehyde, malonic acid, and an organic base thereby forming a Knoevenagel condensation product; and contacting the Knoevenagel condensation product, an oxidant, a metal halide, and optionally an inorganic base thereby forming the butenolide.

The methods described herein exhibits a high degree of functional group tolerance. Consequently, a broad range of butenolides can be prepared. In certain embodiments, the butenolide has Formula 1:

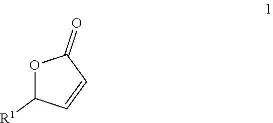

wherein R¹ is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —CN, —OR, —(C═O)OR, —O(C═O)R, —OSiR₃, —O(C═O)OR, —(C═O)NR₂, —(NR)(C═O) R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl. In certain embodiments, the butenolide has Formula 1, wherein R¹ is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —OR, —O(C═O)R, —O(C═O)OR, —OSiR₃, and —(NH)(C═O)OR, wherein R is methyl, ethyl, t-butyl, benzyl, or paramethoxy benzyl. In certain embodiments, R¹ is alkyl, cycloalkyl, aralkyl, aryl, heteroaryl, or —(CR₂)ₘY, wherein m is a whole number selected from 1-10; R for each occurrence is independently selected from the group consisting of hydrogen and alkyl; and Y is selected from the group consisting of —OR, —O(C═O)R, —O(C═O)OR, —OSiR₃, and —(NH)(C═O)OR, wherein R is methyl, ethyl, t-butyl, benzyl, or paramethoxy benzyl.

The aldehyde used in the methods described herein is not particularly limited by the present disclosure, which contemplates all aldehydes having one or two alpha hydrogens. The selection of the appropriate aldehyde is well within the skill of a person of ordinary skill in the art. In certain embodiments, the aldehyde has the formula R¹CH₂CHO, wherein R¹ is alkyl, cycloalkyl, heterocycloalkyl, aralkyl, aryl, heteroaryl, or —(CR₂)ₘY, wherein m is a whole number selected from 1-9; R for each occurrence is independently selected from the group consisting of hydrogen, alkyl, cycloalkyl, heterocycloalkyl, aryl, and heteroaryl; and Y is selected from the group consisting of —CN, —OR, —(C═O)OR, —O(C═O)R, —OSiR₃, —O(C═O)OR, —(C═O)NR₂, —(NR)(C═O)R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl. In certain embodiments, the aldehyde has the formula R¹CH₂CHO₁ wherein R¹ is alkyl, cycloalkyl, aralkyl, aryl, heteroaryl, or —(CR₂)ₘY, wherein m is a whole number selected from 1-9; R for each occurrence is independently selected from the group consisting of hydrogen and alkyl; and Y is selected from the group consisting of —OR, —O(C═O)R, —O(C═O)OR, —OSiR₃, and —(NH)(C═O)OR, wherein R is methyl, ethyl, t-butyl, benzyl, or paramethoxy benzyl.

The organic base can be any base with a sufficient pKb to react with an alpha hydrogen of at least a portion of the malonic acid. The organic base can be a secondary amine, a tertiary amine, a secondary amidine, a tertiary amidine, a secondary guanidine, a tertiary guanidine, and a heteroaromatic amine. In certain embodiments, the organic base has the formula NR²₃, wherein R² for each instance is independently hydrogen or alkyl, wherein no more than one R² is hydrogen; or two instances of R² taken together with the nitrogen to which they are attached form a 3-8 membered heterocycloalkyl optionally containing an additional heteroatom selected from the group consisting of O and N. Exemplary organic bases include, but are not limited to diethyl amine, diisopropyl amine, pyrrolidine, piperidine, hexamethyleneimine, proline, morpholine, piperazine, imidazole, pyridine, pyrazine, trimethylamine, triethylamine, N,N-diisopropylethylamine, N-ethyl pyrrolidine, N-methyl imidazole, N-ethyl morpholine, quinuclidine, 1,4-diazabicyclo [2.2.2] octane (DABCO), 1,8-diazabicyclo[5.4.0]undec-7-ene (DBU), 1,1,3,3-tetramethylguanidine (TMG), 7-methyl-1,5,7-triazabicyclo(4.4.0)dec-5-ene (MTBD), triazabicyclodecene (TBD), and 1,5-diazabicyclo[4.3.0]non-5-ene (DBN). In certain embodiments, the organic base is piperidine.

The step of contacting the aldehyde, malonic acid, and organic base can be conducted in any solvent in which the reagents are at least partially soluble. The selection of the appropriate solvent is well within the skill of a person of ordinary skill in the art. In certain embodiments, the solvent is a polar aprotic organic solvent. alkyl halides, ethers, esters, ketones, carbonates, formamides, alkylnitriles, nitroalkanes, alkylsulfoxides, and aromatic solvents. Exemplary solvents include, but are not limited to, tetrahydrofuran, tetrahydropyran, dioxane, dichloromethane, dichloroethane, chloroform, nitromethane, dimethylformamide, dimethylsulfoxide, propylene carbonate, hexamethylphosphoramide, N-methylpyrrolidinone, sulfolane, and mixtures thereof. In certain embodiments, the step of contacting the aldehyde, malonic acid, and organic base is conducted in dimethylsulfoxide.

The aldehyde and the malonic acid can be contacted in a molar ratio of 1:1 to 1:3, 1:1 to 1:2.5, 1:1 to 1:2, 1:1 to 1:1.9, 1:1 to 1:1.8, 1:1 to 1:1.7, 1:1 to 1:1.6, 1:1 to 1:1.5, 1:1 to 1:1.3, 1:1 to 1:1.2, 1:1 to 1:1.1.

In instances in which the step of contacting the aldehyde, malonic acid, and organic base is contacted in a solvent, the concentration of the aldehyde can range from 0.1-10M, 0.5-10M, 1-10M, 1.25-10M, 3-10M, 4-10M, 5-10M, 6-10M, 7-10M, 8-10M, 9-10M, or 1.25-3M.

The organic base can be used in a catalytic amount, stoichiometric amount, or used in excess relative to the amount of the aldehyde. In certain embodiments, the organic base is present at 0.1-5 mol %, 0.5-5 mol %, 0.5-5 mol %, 1-5 mol %, 1-4 mol %, 1-4 mol %, 1-3 mol %, or 1.5-2.5 mol % relative to the aldehyde. In certain embodiments, the organic base is present at about 2 mol % relative to the aldehyde.

The step of contacting the aldehyde, malonic acid, and organic base can be conducted at a temperature between 23-140° C., 30-140° C., 40-140° C., 50-140° C., 60-140° C., 70-140° C., 80-140° C., 90-140° C., 100-140° C., 50-130° C., 50-120° C., 50-110° C., 50-100° C., 60-100° C., 70-100° C., 80-100° C., 90-100° C., 90-110° C., or 95-105° C.

In certain embodiments, the step of contacting the aldehyde, malonic acid, and organic base is conducted in the presence of acetic acid at a concentration of 0.1-5 mol %, 0.5-5 mol %, 0.5-5mol %, 1-5 mol %, 1-4 mol %, 1-4 mol %, 1-3 mol %, or 1.5-2.5 mol % relative to the aldehyde. The step of contacting the aldehyde, malonic acid, and organic base is conducted in the presence of acetic acid at a concentration of about 2 mol % relative to the aldehyde.

The step of contacting the aldehyde, malonic acid, and organic base can be conducted for a period of time required until substantially all of the aldehyde and/or intermediate products (e.g., β-hydroxy carboxylic acid) have been consumed, the conversion of the aldehyde and/or intermediate products has substantially stopped, and/or the formation of impurities/decomposition products. The reaction can be monitored using any number of methods known in the art, such as thin layer chromatography (TLC), ¹H—NMR, ¹³C—NMR, infrared spectroscopy, or the like. In certain embodiments, the step of contacting the aldehyde, malonic acid, and organic base is conducted from 2-14 hours, 2-12 hours, 2-10 hours, 2-8 hours, 2-6 hours, 2-4 hours, 4-14 hours, 6-14 hours, 8-14 hours, 10-14 hours, 12-14 hours, 4-12 hours, 6-10 hours, 6-8 hours, or 8-10 hours.

In certain embodiments, the step of contacting the aldehyde, malonic acid, and organic base can be conducted in a flow chemistry reactor. Flow chemistry can involve the use of a flow chemistry reactor, wherein one or more chemical reactions run in a continuously flowing stream rather than in a conventional batch reaction. Flow chemistry reactors can involve the use of pumps, which move one or more fluids comprising one or more of reactants through tubes and where two or more tubes join one another and where the fluids and their respective reagents are reactive, a reaction can take place. Flow chemistry is a well-known method that is useful for large scale synthesis of compounds.

In certain embodiments, the step of contacting the aldehyde, malonic acid, and organic base is conducted in flow chemistry reactor in a solvent, wherein the aldehyde, the organic base, and the solvent are each as defined in any embodiment described herein. In certain embodiments, the organic base is piperidine and the solvent comprises dimethyl formamide.

In instances in which the step of contacting the aldehyde, malonic acid, and organic base is conducted in flow chemistry reactor, the aldehyde and the malonic acid can be contacted in a molar ratio of 1:1 to 1:3, 1:1 to 1:2.5, 1:1 to 1:2, 1:1 to 1:1.9, 1:1 to 1:1.8, 1:1 to 1:1.7, 1:1 to 1:1.6, 1:1 to 1:1.5, 1:1 to 1:1.3, 1:1 to 1:1.2, 1:1 to 1:1.1.

In instances in which the step of contacting the aldehyde, malonic acid, and organic base is conducted in flow chemistry reactor, the concentration of the aldehyde can range from 0.1-15M, 1-15M, 5-15M, 6-14M, 7-13M, 8-12M, or 9-11M.

In instances in which the step of contacting the aldehyde, malonic acid, and organic base is conducted in flow chemistry reactor, the organic base is present at 0.1-5 mol %, 0.5-5 mol %, 0.5-5 mol %, 1-5 mol %, 1-4 mol %, 1-4 mol %, 1-3 mol %, or 1.5-2.5 mol % relative to the aldehyde. In certain embodiments, the organic base is present at about 2 mol % relative to the aldehyde.

In instances in which the step of contacting the aldehyde, malonic acid, and organic base is conducted in flow chemistry reactor, the step of contacting the aldehyde, malonic acid, and organic base can be conducted at a temperature between 80-160° C., 80-150° C., 80-140° C., 90-140° C., 100-140° C., 110-140° C., 115-140° C., 115-130° C., 120-140° C., 125-140° C., 130-140° C., or 135-140° C.

In instances in which the step of contacting the aldehyde, malonic acid, and organic base is conducted in flow chemistry reactor, the step of contacting the aldehyde, malonic acid, and organic base can be conducted at a flow rate of 0.1-6.0 mL/min, 0.2-6.0 mL/min, 0.4-6.0 mL/min, 0.8-6.0 mL/min, 1.0-6.0 mL/min, 1.2-6.0 mL/min, 1.5-6.0 mL/min, 2.0-6.0 mL/min, 4.0-6.0 mL/min, 1.0-4.0 mL/min, 1.0-2.0 mL/min, 1.0-1.5 mL/min, 1.0-1.2 mL/min, 0.6-6.0 mL/min, 0.6-4.0 mL/min, 0.6-2.0 mL/min, 0.6-1.5 mL/min, 0.6-1.2 mL/min, 0.6-1.0 mL/min, or 0.6-0.8 mL/min.

The Knoevenagel condensation product can be represented by the Formula 2:

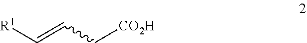

or a conjugate salt thereof, wherein R¹ is as defined in embodiment described herein.

Under certain conditions (e.g., acidic or basic conditions and/or under the oxidative lactonization reaction conditions) the Knoevenagel condensation product may exist in equlibrium with the isomeric form represented by Formula 3:

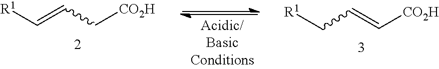

or a conjugate salt thereof, wherein R¹ is as defined herein. In certain embodiments, the compound of Formula 2 is contacted with the oxidant and optionally the inorganic base and undergoes in situ isomerization thereby forming the compound of Formula 3 prior to and/or during the oxidative lactonization.

The oxidant can be a peroxide or oxone. In instances in which the oxidant is a peroxide, the method may not call for an inorganic base. Whereas, when oxidant is oxone, the inorganic base can be present.

The peroxide can be hydrogen peroxide, a peroxycarboxylic acid, an alkyl hydroperoxide, a dialkyl peroxide, or a diacyl hydroperoxide. Exemplary peroxides include, but are not limited to hydrogen peroxide, t-butyl hydroperoxide, di-t-butyl peroxide, dicumyl peroxide, t-butylperoxybenzoate, dibenzoyl peroxide, and peroxyacetic acid. In certain embodiments, the peroxide is hydrogen peroxide.

Oxone, also known as potassium peroxymonosulfate and can be represented by the chemical formula KHSO₅·0.5KHSO₄·0.5K₂SO₄, is a well-known ecofriendly oxidizing agent.

The metal present in the metal halide is not particularly limited and can be any metal. In certain embodiments, the metal halide comprises a Group I or Group II metal, such as Li⁺, Na⁺, K⁺, Rb⁺, Cs⁺, Mg²⁺, Ca²⁺, Sr²⁺, or Ba²⁺. The metal halide can comprise Cl⁻, Br⁻, or I⁻. In certain embodiments, the metal halide is NaBr, NaI, KBr, KI, or mixture thereof.

In instances in which the oxidant is oxone, the step of contacting the Knoevenagel condensation product, oxone, the metal halide, and the inorganic base can be conducted in a solvent comprising acetonitrile, water, or a mixture thereof.

In instances in which the oxidant is oxone, the step of contacting the Knoevenagel condensation product, oxone, the metal halide, and the inorganic base can be conducted in a solvent comprising acetonitrile and water in a volume/volume (v/v) ratio of 20:1 to 1:1, 10:1 to 1:1, 5:1 to 1:1, 2:1 to 1:1, 7:3 to 3:7, 3:2 to 2:3, or 45:55 to 55:45, respectively. In instances in which the oxidant is oxone, the step of contacting the Knoevenagel condensation product, oxone, the metal halide, and the inorganic base can be conducted in a solvent comprising acetonitrile and water in a volume/volume (v/v) ratio of about 1:1, respectively.

In instances in which the oxidant is oxone, the step of contacting the Knoevenagel condensation product, oxone, and the metal halide can be conducted in the presence of an inorganic base. The inorganic base can be a Group I or Group II metal carbonate or bicarbonate. In certain embodiments the inorganic base is Li₂CO₃, Na₂CO₃, K₂CO₃, Cs₂CO₃, MgCO₃, CaCO₃, SrCO₃, or BaCO₃

In instances in which the oxidant is oxone, the inorganic base can be present at 100-200 mol %, 150-200 mol %, or 100-150 mol % relative to the Knoevenagel condensation product.

In instances in which the oxidant is oxone, the metal halide can be present at 0.1-4 equivalents, 1.1-4 equivalents, 1.2-4 equivalents, 2-4, 1.1-2 equivalents, or 1.2-2 equivalents relative to the Knoevenagel condensation product.

In instances in which the oxidant is oxone, the concentration of the Knoevenagel condensation product can range from 0.1-0.5M, 0.1-0.4M, 0.1-0.3M, 0.2-0.5M, 0.3-0.5M, or 0.4-0.5M.

In instances in which the oxidant is oxone, the step of contacting the Knoevenagel condensation product, oxone, the metal halide, and the inorganic base can be conducted at a temperature between −20° C. to 23° C., −10° C. to 23° C., 0° C. to 23° C., −20° C. to 10° C.,-20° C. to 0° C., −10° C. to 10° C., or −5° C. to 5° C.

In instances in which the oxidant is a peroxide, the step of contacting the Knoevenagel condensation product, the peroxide, and the metal halide can be conducted in a solvent comprising acetonitrile, dimethylformamide, tetrahydrofuran, ethyl acetate, water, or a mixture thereof.

In instances in which the oxidant is a peroxide, the step of contacting the Knoevenagel condensation product, the peroxide, and the metal halide can be conducted in a solvent comprising acetonitrile and water in a volume/volume (v/v) ratio of 7:3 to 3:7, 3:2 to 2:3, or 45:55 to 55:45, respectively. In instances in which the oxidant is peroxide, the step of contacting the Knoevenagel condensation product, the peroxide, and the metal halide can be conducted in a solvent comprising acetonitrile and water in a volume/volume (v/v) ratio of about 1:1, respectively.

In instances in which the oxidant is a peroxide, the metal halide can be present at 0.1-1.0, 0.3-1.0, 0.5-1.0, 0.1-0.5, or 0.1-0.3 equivalents relative to the Knoevenagel condensation product. In certain embodiments in which the oxidant is a peroxide, the metal halide is present at about 0.3 equivalents relative to the Knoevenagel condensation product.

In instances in which the oxidant is a peroxide, the peroxide can be present at 1.0-2.0 or 1.0-1.5 equivalents relative to the Knoevenagel condensation product.

In instances in which the oxidant is a peroxide, the concentration of the Knoevenagel condensation product can range from 1-5M, 2-5M, 3-5M, 4-5M, 1-4M, 1-3M, or 1-2M.

In instances in which the oxidant is a peroxide, the step of contacting the Knoevenagel condensation product, the peroxide, and the metal halide can be conducted at a temperature between 0° C. to 50° C., 10° C. to 50° C., 23° C. to 50° C., 23° C. to 40° C., 23° C. to 30° C., or 23° C. to 25° C.

The step of contacting the Knoevenagel condensation product, the oxidant, the metal halide, and optionally the inorganic base can be conducted for a period of time required until substantially all of the Knoevenagel condensation product and/or intermediate products (e.g., β-halo lactones) have been consumed, the conversion of the Knoevenagel condensation product and/or intermediate products has substantially stopped, and/or the formation of impurities/decomposition products. The reaction can be monitored using any number of methods known in the art, such as thin layer chromatography (TLC), ¹H-NMR, ¹³C-NMR, infrared spectroscopy, or the like. In certain embodiments, the step of contacting the Knoevenagel condensation product, the oxidant, the metal halide, and optionally the inorganic base is conducted from 6-48 hours, 12-48 hours, 24-48 hours, 36-48 hours, 12-36 hours, 6-36 hours, or 6-24 hours.

Advantageously, when hydrogen peroxide is used as the oxidant and NaI is the metal halide, the NaI can be isolated after the reaction has completed and reused one or more times without substantially reducing the yield of the butenolide.

As demonstrated in the examples below, the methods described herein are highly scalable and are capable of achieving excellent yields (e.g., 72-87%) of the butenolide on a gram, decagram, and hectogram scale in high purity (e.g., >97% purity) without the need for purification.

### Example 1

### General Procedure A of the Synthesis of Butenolide using Reaction Flask

To a solution of manolic acid (1.1 equiv.) and piperidine (0.1-2 mol %) in DMSO (10 M) was added aldehyde (1 equiv.) slowly. The solution was stirred at room temperature for 30-120 minutes and then heated to 50-100° C. for 2-12 hours. After cooling to room temperature, the solvent was distillated out under reduced pressure. To the crude carboxylic acid intermediate dissolved in an organic solvent (DMF, MeCN, THF, or EtOAc) was added Group I or Group II metal halide (0.01-1.0 equiv.) and hydrogen peroxide (1.5-3.5 equiv.) or oxone (1.2-2 equiv.) [Caution: It is an exothermic reaction. Slow addition of H₂O₂ in water bath]. The reaction was stirred at room temperature for 6-48 h. Na₂SO₃ was added to the reaction mixture until a clear solution was observed (decolor). The organic solvent was removed under reduced pressure. Extraction of remaining aqueous solution with ethyl acetate (2 times). The combined organic fraction was washed with brine, dried over anhydrous MgSO₄, filtered and concentrated under reduced pressure to obtain the desired product. Distillate solvents and aqueous phase after extraction are recyclable.

### Example 2

### General Procedure B of the Synthesis of Butenolide Using Flow Reactor

To a solution of manolic acid (1.1 equiv.) and piperidine (0.1-2 mol %) in DMF or DMSO (10 M) was added aldehyde (1 equiv.) slowly. The solution was stirred at room temperature for 30-120 minutes and then injected to flow reactor preheated to 100-140° C. at a flow rate of 0.4-6.0 mL/min. The solvent of collected solution was distilled out under reduced pressure. To the crude carboxylic acid intermediate dissolved in an organic solvent (DMF, MeCN, THF, or EtOAc) was added Group I or Group II metal halide (0.01-1.0 equiv.) and hydrogen peroxide (1.5-3.5 equiv.) or oxone (1.2-2 equiv.) [Caution: It is an exothermic reaction. Slow addition of H₂O₂ in water bath]. The reaction was stirred at room temperature for 6-48 h. Na₂SO₃ was added to the reaction mixture until a clear solution was observed (decolor). The organic solvent was removed under reduced pressure. Extraction of remaining aqueous solution with ethyl acetate (2 times). The combined organic fraction was washed with brine, dried over anhydrous MgSO₄, filtered and concentrated under reduced pressure to obtain the desired product. Distillate solvents and aqueous phase after extraction are recyclable.

### Example 3

### Synthesis of Butenolide

According to the General Procedure A, decanal (1.56 g, 10 mmol) was used to provide the desired butenolide 1 as yellow oil (1.48 g, 75%). Decagram synthesis from decanal (83 g, 0.53 mol) delivered 76.2 g butenolide 1 (73%). Hectogram synthesis from decanal (415 g, 2.66 mol) provided 374.8 g butenolide 1 (72%, purity >97%). According to the General Procedure B, decanal (10.45 g, 67 mmol) was used to provide the desired butenolide 1 (11.4 g, 87%). ¹H NMR (400 MHZ, CDCl₃) δ 7.44 (dd, J=5.7, 1.5 Hz, 1H), 6.07 (dd, J=5.7, 2.0 Hz, 1H), 5.01 (ddt, J=7.2, 5.3, 1.8 Hz, 1H), 1.80-1.56 (m, 2H), 1.40 (m, 2H), 1.32-1.15 (m, 10H), 0.84 (t, J=7.1 Hz, 3H); ¹³C NMR (100 MHZ, CDCl₃) δ 173.4, 156.5, 121.5, 83.6, 33.2, 31.9, 29.4, 29.4, 29.2, 25.0, 22.7, 14.1; HRMS (ESI) m/z: Calculated C₁₂H₂₀O₂Na⁺ [M+Na]⁺ 219.1356; Found 219.1356.

### Example 4-5

### hexylfuran-2(5H)-one

According to the General Procedure A, octanal (1.28 g, 10 mmol) was used to synthesize 5-hexylfuran-2 (5H)-one (butenolide 2) as pale yellow oil (1.22 g, 73%). ¹H NMR (400 MHz, CDCl₃) δ 7.45 (dd, J=5.7, 1.5 Hz, 1H), 6.09 (dd, J=5.7, 2.0 Hz, 1H), 5.03 (ddt, J=7.3, 5.3, 1.8 Hz, 1H), 1.80-1.70 (m, 1H), 1.70-1.56 (m, 1H), 1.49-1.36 (m, 2H), 1.38-1.15 (m, 6H), 0.87 (t, J=6.9 Hz, 3H); ¹³C NMR (100 MHz, CDCl₃) δ 173.4, 156.5, 121.6, 83.6, 33.3, 31.7, 29.1, 25.0, 22.6, 14.1; HRMS (ESI) m/z: Calculated C₁₀H₁₆O₂Na⁺ [M+Na]⁺ 191.1043; Found 191.1035.

### Example 5

### Syntheis of 5-(8-hydroxyoctyl)furan-2(5H)-one

According to the General Procedure A, tetrahydropyran protected 10-hydroxydacanal was prepared by two steps sequence from 1,10-decanediol (871 mg, 5 mmol) and used for the synthesis of 5-(8-hydroxyoctyl)furan-2(5H)-one. The crude intermediate from oxidative lactionalization was dissolved in methanol (5 mL) and added protic acid and stirred for 6 h. The acid was removed by filtration and the filtrate was concentrated under reduced pressure to obtain the 5-(8-hydroxyoctyl)furan-2(5H)-one (butenolide 3) as pale yellow solid (368 mg, 34% over 5 steps). Melting point 33-35° C.; ¹H NMR (400 MHZ, CDCl₃) δ 7.44 (dd, J=5.7, 1.5 Hz, 1H), 6.09 (dd, J=5.7, 2.0 Hz, 1H), 5.02 (ddt, J=7.2, 5.2, 1.8 Hz, 1H), 3.62 (t, J=6.6 Hz, 2H), 1.81-1.70 (m, 1H), 1.70-1.59 (m, 2H), 1.54 (m, 2H), 1.48-1.38 (m, 1H), 1.38-1.22 (m, 9H); ¹³C NMR (100 MHZ, CDCl₃) δ 173.4, 156.5, 121.6, 83.6, 63.1, 33.2, 32.8, 29.4, 29.3, 29.3, 25.8, 25.0; HRMS (ESI) m/z: Calculated C₁₂H₂₀O₃Na⁺ [M+Na]⁺ 235.1305; Found 235.1303.

### Example 6

### Synthesis of 5-(10-hydroxydecyl)furan-2(5H)-one

According to the General Procedure A, the crude tetrahydropyran protected 10-hydroxydacanal was prepared by two steps sequence from 1,12-dodecanediol (1.01 g, 5 mmol) and used for the synthesis of 5-(10-hydroxydecyl)furan-2(5H)-one. The crude intermediate from oxidative lactionalization was dissolved in methanol (5 mL) and added protic acid and stirred for 6 h. The protic acid was removed by filtration and the filtrate was concentrated under reduced pressure to obtain the 5-(10-hydroxydecyl)furan-2(5H)-one (butenolide 4) as pale yellow solid (442 mg, 37% over 5 steps). Melting point 46-48° C.; ¹H NMR (400 MHZ, CDCl₃) δ 7.44 (dd, J=5.7, 1.5 Hz, 1H), 6.10 (dd, J=5.8, 2.0 Hz, 1H), 5.03 (ddt, J=7.3, 5.3, 1.8 Hz, 1H), 3.63 (t, J=6.6 Hz, 2H), 1.82-1.70 (m, 1H), 1.70-1.60 (m, 1H), 1.60-1.52 (m, 2H), 1.49 (s, 1H), 1.46-1.39 (m, 2H), 1.39-1.20 (m, 12H); ¹³C NMR (100 MHz, CDCl₃) δ 173.3, 156.5, 121.7, 83.6 63.2, 33.3, 32.9, 29.6, 29.5 (2C), 29.4, 29.4, 25.8, 25.1; HRMS (ESI) m/z: Calculated C₁₄H₂₄O₃Na⁺ [M+Na]⁺ 263.1618; Found 263.1618.

What is claimed is:

1. A method of preparing a butenolide, the method comprising: contacting an aldehyde, malonic acid, and an organic base thereby forming a Knoevenagel condensation product; and contacting the Knoevenagel condensation product, an oxidant, a metal halide, and optionally an inorganic base thereby forming the butenolide.

2. The method of claim 1, wherein the organic base is an organic amine, an organic amidine, or an organic guanidine.

3. The method of claim 1, wherein the oxidant is a peroxide or oxone.

4. The method of claim 1, wherein the metal halide is a Group I or Group II metal salt of chloride, bromide, or iodide.

5. The method of claim 1, wherein the butenolide has Formula 1: 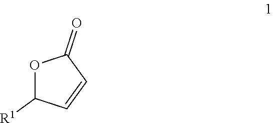
wherein R¹ is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —CN, —(C═O)OR, —OR, —O(C═O)R, —OSiR³, —O(C═O)OR, —(C═O)NR₂, —(NR)(C═O)R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl.

6. The method of claim 5, wherein the organic base is an organic amine.

7. The method of claim 6, wherein the organic base is selected from the group consisting of diethyl amine, diisopropyl amine, pyrrolidine, piperidine, hexamethyleneimine, proline, morpholine, piperazine, imidazole, pyridine, triethylamine, N,N-diisopropylethylamine, N-ethyl pyrrolidine, N-nethyl morpholine, quinuclidine, and 1,4-diazabicyclo[2.2.2]octane.

8. The method of claim 6, wherein the organic base comprises piperidine.

9. The method of claim 8, wherein the step of contacting the aldehyde, malonic acid, and piperidine is conducted in a solvent comprising dimethyl sulfoxide, dimethyl formamide, or a mixture thereof; and piperidine is present at 0.5-3 mol % relative to the aldehyde.

10. The method of claim 8, wherein the step of contacting the aldehyde, malonic acid, and piperidine is conducted in a solvent comprising dimethyl sulfoxide at 50-100° C.

11. The method of claim 8, wherein the step of contacting the aldehyde, malonic acid, and piperidine is conducted in a flow chemistry reactor in a solvent comprising dimethyl sulfoxide, dimethyl formamide, or a mixture thereof at 100-140° C.

12. The method of claim 11, wherein the flow chemistry reactor is operated at a flow rate of 0.4 mL/min to 6.0 mL/min.

13. The method of claim 5, wherein the oxidant comprises oxone; the metal halide comprises a Group I metal salt of iodide; and the inorganic base comprises a Group I or Group II metal carbonate or bicarbonate.

14. The method of claim 13, wherein the inorganic base comprises sodium carbonate, the metal halide comprises potassium iodide, and the step of contacting the Knoevenagel condensation product, oxone, the potassium iodide, and sodium carbonate is conducted in a solvent comprising acetonitrile and water in a volume ratio of 2:1 to 1:2, respectively.

15. The method of claim 14, wherein the step of contacting the Knoevenagel condensation product, oxone, potassium iodide, and sodium carbonate is conducted at −10 to 23° C.

16. The method of claim 5, wherein the oxidant comprises hydrogen peroxide; and the metal halide comprises a Group I or Group II metal bromide or iodide.

17. The method of claim 16, wherein the metal halide comprises sodium iodide and the step of contacting the Knoevenagel condensation product, hydrogen peroxide, and sodium iodide is conducted in a solvent comprising acetonitrile and water in a volume ratio of 2:1 to 1:2, respectively.

18. The method of claim 17, wherein the step of contacting the Knoevenagel condensation product, hydrogen peroxide, and sodium iodide is conducted at −10 to 23° C.

19. The method of claim 1, wherein the method comprises:
contacting an aldehyde having the formula R¹CH₂CHO, malonic acid and piperdine in a solvent comprising dimethyl sulfoxide at 50-100° C., wherein piperidine is present at 0.5-3 mol % relative to the aldehyde; or
contacting an aldehyde having the formula R¹CH₂CHO, malonic acid, and piperdine in a solvent comprising dimethyl formamide in a flow chemistry reactor at a temperature of 100-140° C. and a flow rate of 0.4 mL/min to 6.0 mL/min;
thereby forming the Knoevenagel condensation product; and
contacting the Knoevenagel condensation product, oxone, potassium iodide, and sodium carbonate in a solvent comprising acetonitrile and water in a volume ratio of 2:1 to 1:2, respectively at −10 to 23° C.; or
contacting the Knoevenagel condensation product, hydrogen peroxide, and sodium iodide in a solvent comprising acetonitrile and water in a volume ratio of 2:1 to 1:2, respectively at −10 to 23° C.;
thereby forming the butenolide, wherein R¹ is is alkyl, cycloalkyl, heterocycloalkyl, aralkyl, aryl, heteroaryl, or —(CR₂)ₘY, wherein m is a whole number selected from 1-10; R for each occurrence is independently selected from the group consisting of hydrogen, alkyl, cycloalkyl, heterocycloalkyl, aryl, and heteroaryl; and Y is selected from the group consisting of —CN, —(C═O)OR, —O(C═O)R, —O(C═O)OR, —(C═O)NR₂, —(NR)(C═O)R, —(NR)(C═O)OR, —O(C═O)NR₂, —O(C═NR)NR₂, —(NR)(C═O)NR₂, —(S═O)R, —S(O)₂R, —S(O)₂OR, —S(O)₂NR₂, —OS(O)₂R, —(NR)S(O)₂R, and —(NR)S(O)₂NR₂, wherein R for each instance is independently hydrogen, alkyl, cycloalkyl, heterocycloalkyl, aralkyl, aryl, or heteroaryl.

20. The method of claim 19, wherein the method comprises: contacting the aldehyde having the formula R¹CH₂CHO, malonic acid, and piperdine in a solvent comprising dimethyl formamide in a flow chemistry reactor at a temperature of 100-140° C. and a flow rate of 0.4 mL/min to 6.0 mL/min thereby forming the Knoevenagel condensation product; and
contacting the Knoevenagel condensation product, oxone, potassium iodide, and sodium carbonate in a solvent comprising acetonitrile and water in a volume ratio of 2:1 to 1:2, respectively at −10 to 23° C.; or
contacting the Knoevenagel condensation product, hydrogen peroxide, and sodium iodide in a solvent comprising acetonitrile and water in a volume ratio of 2:1 to 1:2, respectively at −10 to 23° C. thereby forming the butenolide.
